(19) United States
(12) Patent Application Publication
(10) Pub. No.: US 2016/0183772 A1
(43) Pub. Date: Jun. 30, 2016

(54) MEDICAL DEVICE

(71) Applicant: OLYMPUS CORPORATION, Tokyo (JP)

(72) Inventor: Izumi HATTA, Tokyo (JP)

(73) Assignee: OLYMPUS CORPORATION, Tokyo (JP)

(21) Appl. No.: 14/811,084

(22) Filed: Jul. 28, 2015

Related U.S. Application Data
(63) Continuation of application No. PCT/JP2014/051600, filed on Jan. 21, 2014.
(60) Provisional application No. 61/757,801, filed on Jan. 29, 2013.

(51) Int. Cl. A61B 1/00

(52) U.S. Cl. CPC A61B 1/00133; A61B 1/00087

(57) ABSTRACT

A medical device including a long main body having a distal end portion; two or more movable portions arranged at the main body or in a vicinity of the distal end portion; a single input interface part operated by an operator; and a control part switching and controlling the respective movable portions in accordance with an operation amount input to the input interface part.

### CROSS-REFERENCE TO RELATED APPLICATIONS

This is a continuation of International Application PCT/JP2014/051600, with an international filing date of Feb. 21, 2014, which is hereby incorporated by reference herein in its entirety. This application claims the benefit of Provisional Patent Application No. 61/757,801, the content of which is incorporated herein by reference.

### TECHNICAL FIELD

The present invention relates to a medical device.

### BACKGROUND ART

A known treatment tool system in the related art drives a medical device such as an endoscope in which a treatment tool is made to protrude from the distal end (for example, see PTL 1).

This treatment tool system separately includes a joystick for operating bending of a bending portion of the endoscope, and a treatment tool driving means for driving the treatment tool.

### CITATION LIST

### Patent Literature

{PTL 1} the Publication of Japanese Patent No. 4580973

### SUMMARY OF INVENTION

An aspect of the present invention provides a medical device comprising: a long main body including a distal end portion; two or more movable portions arranged at the main body or in a vicinity of the distal end portion; a single input interface part operated by an operator; and a control part switching and controlling the respective movable portions in accordance with an operation amount input to the input interface part.

### BRIEF DESCRIPTION OF DRAWINGS

FIG. 1 is an entire configuration diagram illustrating a medical device according to one embodiment of the present invention.

FIG. 2 shows one example of an input interface part of the medical device in FIG. 1.

FIG. 3(a) shows a state where the input interface part in FIG. 2 is positioned at an origin position, FIG. 3(b) shows an endoscope image at that time, and FIG. 3(c) shows states of an endoscope and a treatment tool at that time.

FIG. 4(a) shows a state where the input interface part in FIG. 2 is positioned in a treatment tool movable region, FIG. 4(b) shows an endoscope image at that time, and FIG. 4(c) shows states of the endoscope and the treatment tool at that time.

FIG. 5(a) shows a state where the input interface part in FIG. 2 is positioned in an endoscope movable region, and an operation amount is increased, FIG. 5(b) shows an endoscope image at that time, and FIG. 5(c) shows states of the endoscope and the treatment tool at that time.

FIG. 6(a) shows a state where the input interface part in FIG. 2 is positioned in the endoscope movable region, and an operation amount is decreased, FIG. 6(b) shows an endoscope image at that time, and FIG. 6(c) shows states of the endoscope and the treatment tool at that time.

FIG. 7(a) shows a state where the input interface part in FIG. 2 is positioned in the treatment tool movable region, FIG. 7(b) shows an endoscope image at that time, and FIG. 7(c) shows states of the endoscope and the treatment tool at that time.

FIG. 8 is a flowchart illustrating a control method for the medical device in FIG. 1.

FIG. 9(a) shows an example of the treatment tool movable region and the endoscope movable region of the medical device in FIG. 1, and FIG. 9(b) shows an example of the treatment tool movable region and the endoscope movable region of a modified example of the medical device in FIG. 1.

FIG. 10 is a block diagram illustrating a modified example of the medical device in FIG. 1.

FIG. 11 is a block diagram illustrating another modified example of the medical device in FIG. 1.

FIG. 12(a) is a graph representing a case where ratios between operation of the bending movable portions of the treatment tool and the endoscope and operation input according to another modified example of the medical device in FIG. 1 are linearly switched, FIG. 12(b) is a graph representing a case where ratios between operation of the bending movable portions of the treatment tool and the endoscope and operation input according to another modified example of the medical device in FIG. 1 are kept constant, and FIG. 12(c) is a graph representing a case where ratios between operation of the bending movable portions of the treatment tool and the endoscope and operation input according to another modified example of the medical device in FIG. 1 are non-linearly switched.

### DESCRIPTION OF EMBODIMENT

The following describes a medical device 1 according to one embodiment of the present invention with reference to the drawings.

As shown in FIG. 1, the medical device 1 according to the present embodiment includes an endoscope (main body) 2 including an object optical system 2b at a distal end surface 2a, a treatment tool 3 arranged so as to protrude from the distal end surface 2a via a forceps channel 2c of the endoscope 2, an input IF (input interface part) 4 operated by an operator, and a control part 5 controlling the endoscope 2 and the treatment tool 3 by switching the endoscope 2 and the treatment tool 3 from each other on the basis of an operation amount input to the input IF 4.

The endoscope 2 includes a first bending movable portion (movable portion) 2A that bends the endoscope 2 so as to swivel the distal end surface 2a on a slightly proximal end side compared with the distal end surface 2a in a direction intersecting with the axis. The treatment tool 3 includes an end effector 3a such as grasping forceps at the distal end thereof, a columnar body portion 3b on the proximal end side thereof, and a second bending movable portion (movable portion) 3A that is provided at an intermediate position in the longitudinal direction of the body portion 3b and that bends the body portion 3b so as to swivel the end effector 3a in a direction intersecting with the axis. The first and second bending movable portions 2A and 3A respectively bend in two axial directions, and combination of the bending enables a position of the end effector 3a to be changed in respective two dimensions.

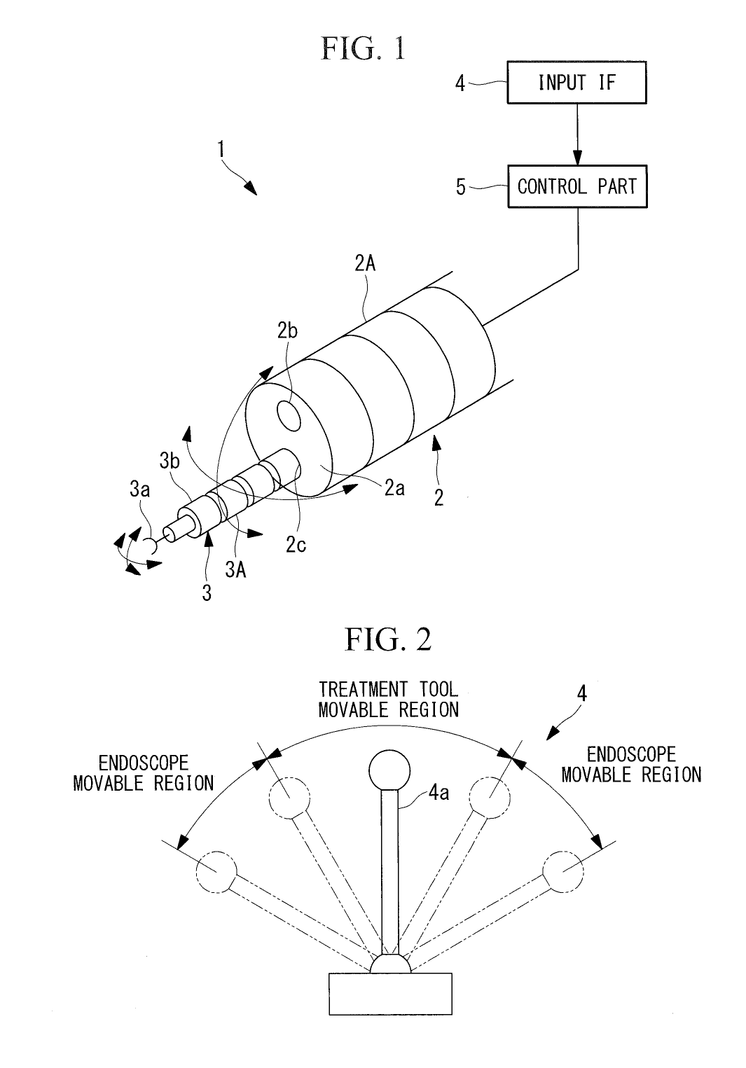

The input IF 4 is a joystick, for example. As shown in FIG. 2, the input IF 4 outputs, to the control part 5, a signal depending on a swiveling direction of a lever 4a and magnitude of a swiveling angle (operation amount) of the lever 4a. The lever 4a of the input IF 4 is provided so as to be swivelable in all directions of 360° from the origin position depicted by the solid line in FIG. 2.

The control part 5 stores swiveling directions of the lever 4a of the input IF 4 and bending directions of the bending movable portions 2A and 3A of the endoscope 2 and the treatment tool 3 in association with each other. When the lever 4a is made to swivel, the control part 5 drives one of the bending movable portions 2A and 3A in the direction stored in association with the swiveling direction by an angle depending on a swiveling angle of the lever 4a.

In the present embodiment, the control part 5 drives only the second bending movable portion 3A of the treatment tool 3 in a treatment tool movable region that is a predetermined angle range including the origin position of the lever 4a. The control part 5 drives only the first bending movable portion 2A of the endoscope 2 in an endoscope movable range that is a predetermined angle range outside the treatment tool movable region.

After a swiveling angle of the lever 4a increases and reaches the endoscope movable region, when operation is performed in a direction of increasing a swiveling angle, the control part 5 drives the first bending movable portion 2A of the endoscope 2. After the swiveling angle of the lever 4a increases and reaches the endoscope movable region, when operation is performed in a direction of decreasing a swiveling angle, the control part 5 makes drive of the first bending movable portion 2A of the endoscope 2 invalid.

A control method for the thus-configured medical device 1 according to the present embodiment is described below.

In order to treat an affected part A by using the medical device 1 according to the present embodiment, the endoscope 2 is inserted into a body of a patient, and while watching an image of an inside of the body obtained via the object optical system 2b, the distal end surface 2a of the endoscope 2 is made to face the affected part A, and the treatment tool 3 is introduced via the forceps channel 2c to be made to protrude from the distal end surface 2a of the endoscope 2.

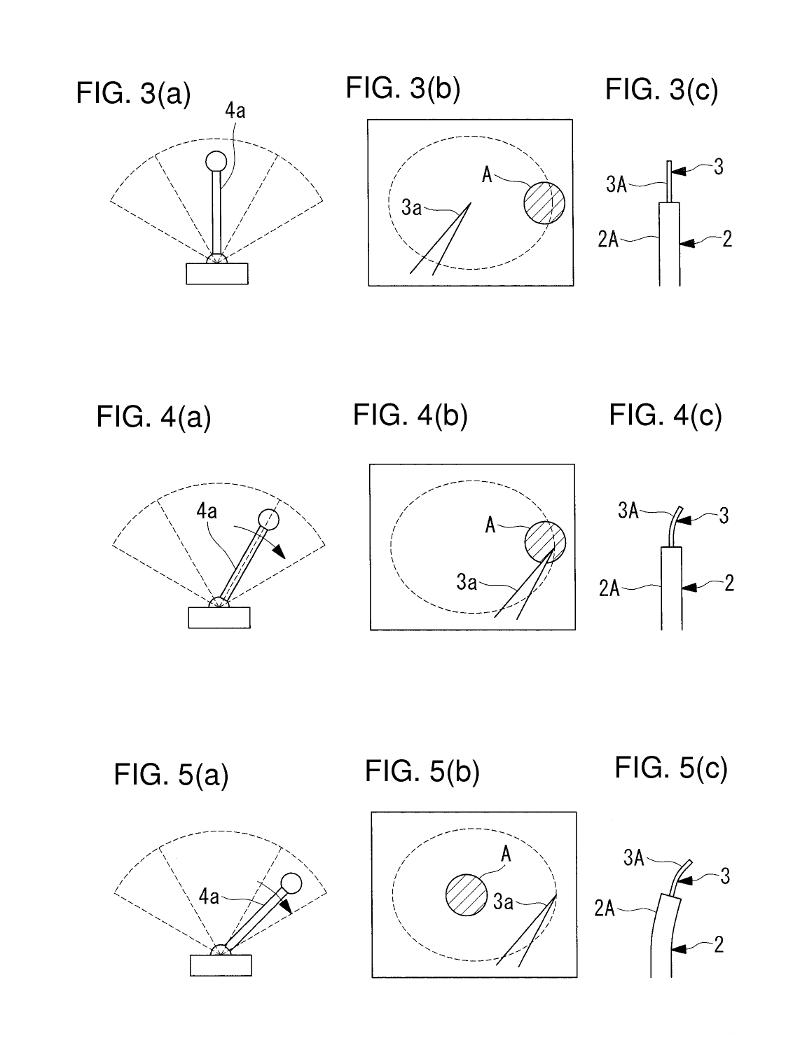

Thereby, as shown in FIG. 3(b), in an image obtained via the object optical system 2b, the affected part A and the end effector 3a at the distal end of the treatment tool 3 are observed. At this time, for example, the lever 4a of the input IF 4 is arranged at the origin position as shown in FIG. 3(a). Accordingly, each of the first bending movable portion 2A of the endoscope 2 and the second bending movable portion 3A of the treatment tool 3 does not bend, and has a shape extending straight as shown in FIG. 3(c).

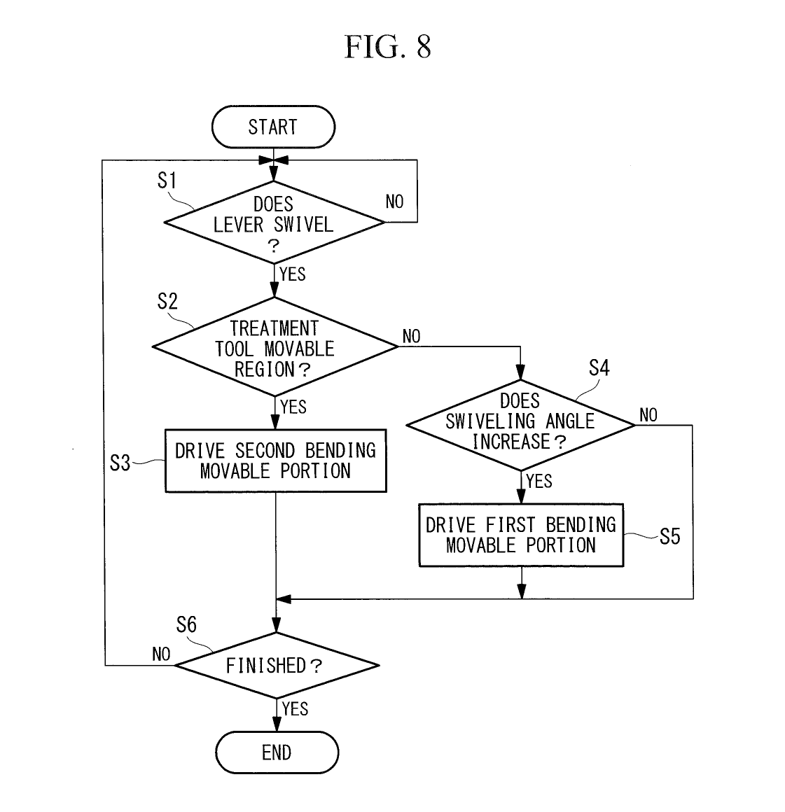

When an operator such as a medical doctor operates the lever 4a of the input IF 4, as shown in FIG. 8, the control part 5 determines whether or not the lever 4a is made to swivel (step S1). When the lever 4a is not made to swivel, the control part 5 waits until the lever 4a is made to swivel. When the lever 4a is made to swivel, it is determined whether or not a swiveling angle stays in the treatment tool movable region (step S2). When the swiveling angle stays in the treatment tool movable region, the second bending movable portion 3A of the treatment tool 3 is made to bend by an angle depending on a swiveling angle of the lever 4a (step S3).

At this time, as shown in FIG. 4(a), the lever 4a of the input IF 4 is positioned in the treatment tool movable region. Accordingly, in a state where a visual field range of the endoscope 2 is fixed, only the end effector 3a of the treatment tool 3 moves in an image shown in FIG. 4(b). In other words, as shown in FIG. 4(c), while the first bending movable portion 2A of the endoscope 2 does not bend, only the second bending movable portion 3A of the treatment tool 3 has a bent shape.

Meanwhile, when a swiveling angle increases to be outside the treatment tool movable region, drive of the second bending movable portion 3A of the treatment tool 3 is stopped, and it is determined whether or not the swiveling angle increases (step S4). When a swiveling angle increases, only the first bending movable portion 2A of the endoscope 2 is made to bend by an angle depending on a swiveling angle increase amount of the lever 4a from the time that the lever 4a of the input IF 4 enters the endoscope movable region.

In other words, as shown in FIG. 5(a), when a swiveling angle is increased with the lever 4a being positioned in the endoscope movable region, the first bending movable portion 2A of the endoscope 2 is driven. For this reason, a position of the distal end surface 2a of the endoscope 2 including the object optical system 2b is changed to change a visual field range as shown in FIG. 5(b). The second bending movable portion 3A of the treatment tool 3 does not change. For this reason, relative position relation between the treatment tool 3 and the object optical system 2b does not change, and only the background moves in a state where the treatment tool 3 is fixed in a visual field of the endoscope 2. At this time, as shown in FIG. 5(c), both of the first bending movable portion 2A of the endoscope 2 and the second bending movable portion 3A of the treatment tool 3 take shapes bending in the same direction.

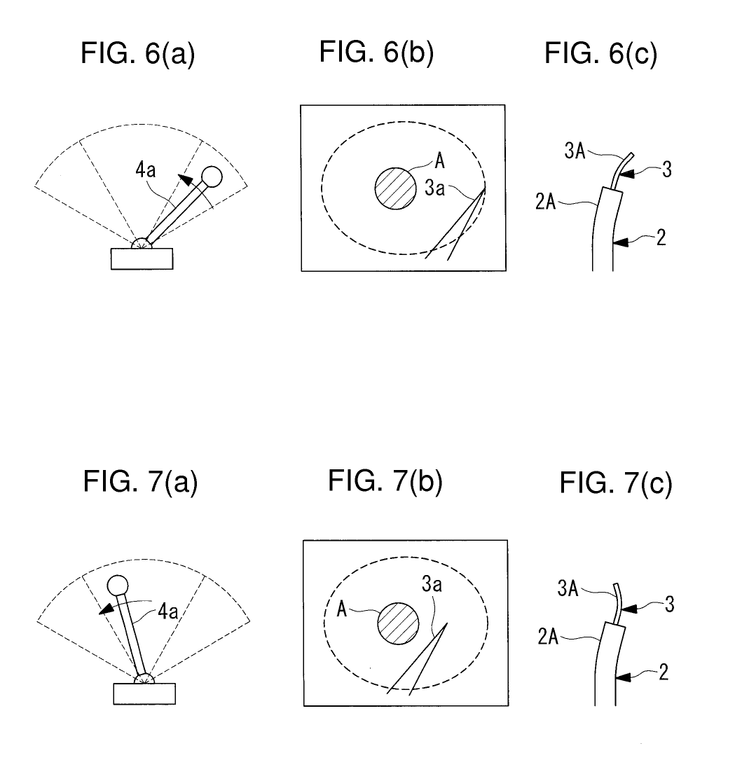

When the affected part A is arranged at a desired position in the visual field range of the endoscope 2, for example, when the affected part A is arranged in the vicinity of the center of the visual field, an operator moves the lever 4a in the returning direction as shown in FIG. 6(a). Thereby, a swiveling angle of the lever 4a is decreased so that drive of the first bending movable portion 2A is stopped. In other words, even if the lever 4a is positioned in the endoscope movable region, both of the bending movable portions 2A and 3A are put into an un-driven state.

As shown in FIG. 7(a), after a swiveling angle of the lever 4a decreases to enter the treatment tool movable region, the second bending movable portion 3A is driven (step S3). In other words, the second bending movable portion 3A of the treatment tool 3 is driven in the direction of decreasing a swiveling angle, and as shown in FIG. 7(b), the end effector 3a moves in the opposite direction in the fixed visual field. Then, as shown in FIG. 7(a), the lever 4a is made to swivel beyond the origin position in the opposite direction. Thereby, the second bending movable portion 3A of the treatment tool 3 takes a shape bent in the opposite direction as shown in FIG. 7(c).

Thus, according to the medical device 1 of the present embodiment, the following advantages are obtained. The two bending movable portions 2A and 3A of the endoscope 2 and the treatment tool 3 are seamlessly driven by the single input IF 4. Operation of making switching between modes does not need to be performed, and troublesomeness of the operation can be resolved.

For the present embodiment, the joystick is described as an example of the input IF 4. However, the input IF 4 is not limited to this, and a three-dimensional haptic device such as PHANToM made by SensAble Technologies Inc. in the United States of America, or a trackball may be used.

In the case of the three-dimensional haptic device, a position of the end effector may be determined in accordance with a position of a distal end of an operated stick so that drive of the bending movable portions 2A and 3A of the endoscope 2 and the treatment tool 3 is switched from each other in accordance with a position of the distal end of the stick. Alternatively, in the case of the three-dimensional haptic device, an angle of the end effector may be determined by orientation of the stick so that drive of the bending movable portions 2A and 3A of the endoscope 2 and the treatment tool 3 is switched from each other in accordance with magnitude of an angle of the stick.

In the case of the trackball, drive of the bending movable portions 2A and 3A of the endoscope 2 and the treatment tool 3 is switched from each other in accordance with a rotational angle of the trackball.

When switching between the treatment tool movable region and the endoscope movable region occurs during operation of the input IF 4, an informing means (not shown) may be provided to inform an operator of this event. This informing may be performed by displaying a message on a screen, changing display on a screen, or giving vibration or click sense to the joystick, for example.

Further, in the present embodiment, a swiveling angle range of the lever 4a of the joystick is divided such that the divided regions are allocated as movable regions for the two bending movable portions 2A and 3A, and the bending movable portions 2A and 3A are respectively driven in accordance with a swiveling angle of the lever 4a. However, instead of this, a swiveling angle range of the lever 4a may be divided such that the divided regions are allocated as movable regions for the two bending movable portions 2A and 3A, the bending movable portion 3A of the treatment tool 3 is driven by an angle depending on a swiveling angle of the lever 4a in the treatment tool movable region, and the endoscope 2 is moved in a certain direction at a speed depending on a swiveling angle of the lever 4a in the endoscope movable region.

In this manner, on the assumption that a swiveling angle of the lever 4a varies in the range up to 60°, for example, in the case of position input, a range of 0° to 30° is allocated to the treatment tool operation region, and a range of 30° to 60° is allocated to the endoscope operation region. However, in the case of speed input for the endoscope operation region, only keeping the lever 4a inclined can continue to move the endoscope 2, and the endoscope operation region can be made narrower than in the case of position input.

Accordingly, position input is performed in the treatment tool operation region, and speed input is performed in the endoscope operation region so that an advantage can be obtained in that a range of 0° to 55° can be allocated to the treatment tool operation region, and a range of 55° to 60° can be allocated to endoscope operation region, for example, and more precise operation is enabled.

In the present embodiment, the two bending movable portions 2A and 3A of the endoscope 2 and the treatment tool 3 are controlled by the single input IF 4. However, instead of this, two or more other arbitrary bending movable portions may be controlled.

For example, examples of the bending movable portions may include a forceps raising base and the endoscope 2, a plurality of joints of the endoscope 2, the endoscope 2 and an over-tube that includes a driving part and into which the endoscope 2 is inserted, the endoscope 2 and the treatment tool 3 provided at the distal end thereof so as to protrude, the endoscope 2 and an arm including an end effector at the distal end thereof, or combination of them and the endoscope 2 including a plurality of joints.

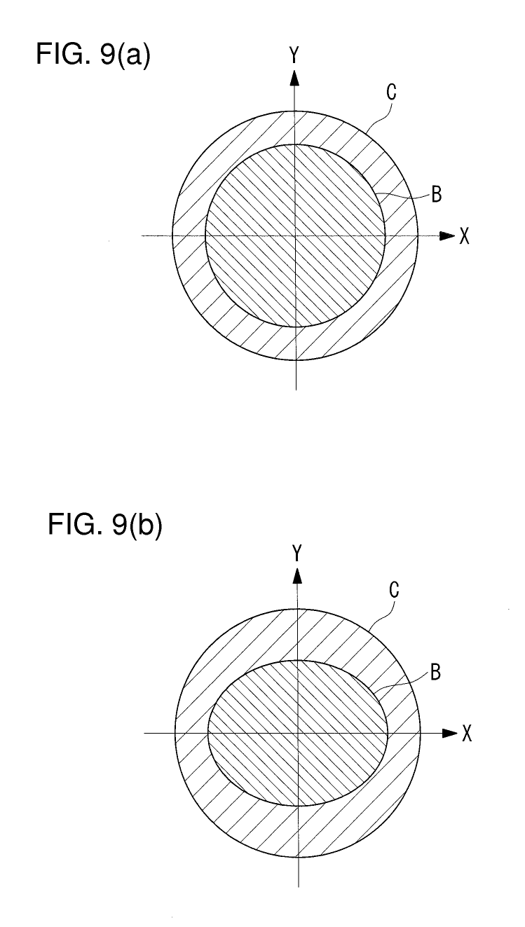

Further, in the present embodiment, a swiveling angle range of the lever 4a is divided into the treatment tool movable region and the endoscope movable region. However, there is a case where movable regions of the treatment tools 3 vary depending on the treatment tools 3. For this reason, preferably, a movable region is set for each treatment tool 3. For example, in one case, as shown in FIG. 9(a), a movable region B of the treatment tool 3 is uniform over all directions with the origin position being the center, and in another case, as shown in FIG. 9(b), a movable region B of the treatment tool 3 is non-uniform (elliptic). In the drawing, the reference sign C denotes a movable region of the endoscope 2.

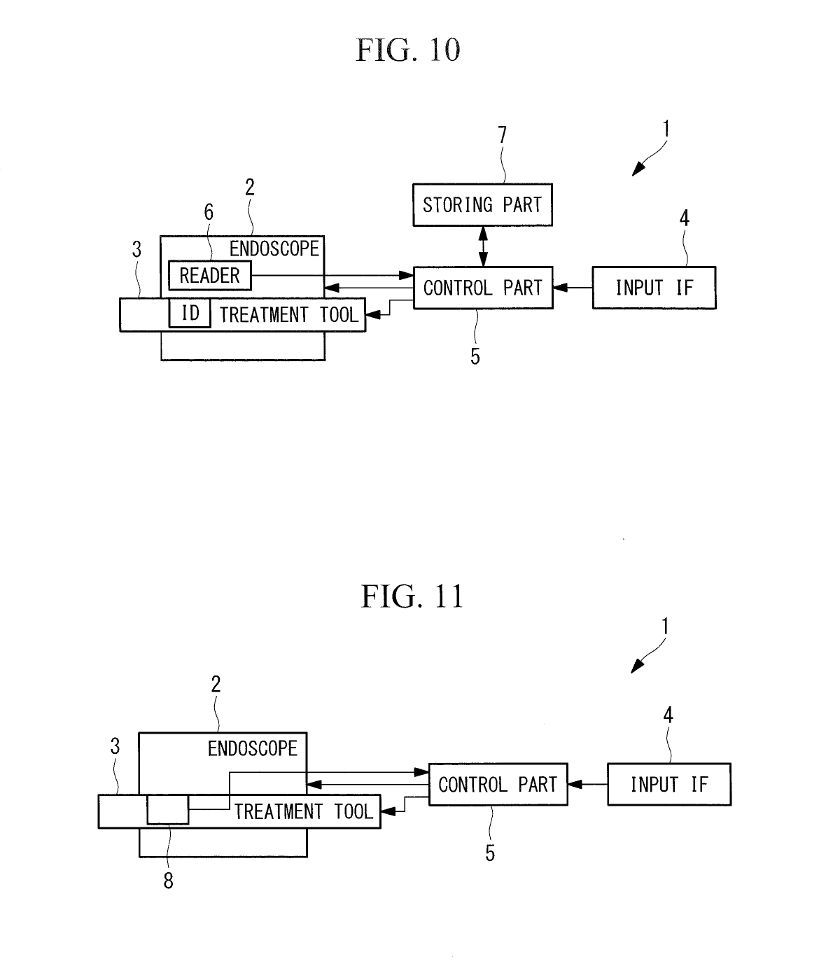

As shown in FIG. 10, the respective treatment tool 3 may include respective identification information (ID), the endoscope 2 may include a reader 6 that reads the identification information, and a storing part 7 that stores the identification information and information of the movable regions in association with each other may be connected to the control part 5. With this configuration, the control part 5 uses, as a key, the identification information read by the reader 6 to read, from the storing part 7, the corresponding information of the movable region so that the treatment tool movable region and the endoscope movable region can be set, and appropriate operation can be performed in accordance with the treatment tool 3.

In the above, the bending movable portions 2A and 3A to be driven are switched from each other in accordance with a swiveling angle of the lever 4a of the joystick. However, instead of this, a movable region limit (a motion limit) of the second bending movable portion 3A of the treatment tool 3 may be detected so that when the movable region limit is not detected, the second bending movable portion 3A of the treatment tool 3 is driven, and when the movable region limit is detected, the first bending movable portion 2A of the endoscope 2 is driven.

As a method of detecting the movable region limit, there is a method in which a sensor 8 detecting a degree of bending of the second bending movable portion 3A is provided as shown in FIG. 11 to directly detect the movable region limit. Alternatively, an image obtained by the endoscope 2 may be processed so that the treatment tool 3 in the image is recognized, and from a position of the treatment tool 3 in the image, the movable region limit is detected.

The method of thus detecting the movable region limit by the sensor 8 or the image process is adopted so that a plurality of bending movable portions can be operated by the single input IF 4 without switching between modes, not only in a case of using the joystick, PHANToM made by SensAble Technologies Inc. in the United States of America, or the track ball, but also in a case of using the input IF 4 including push buttons arranged right and left and backward and forward.

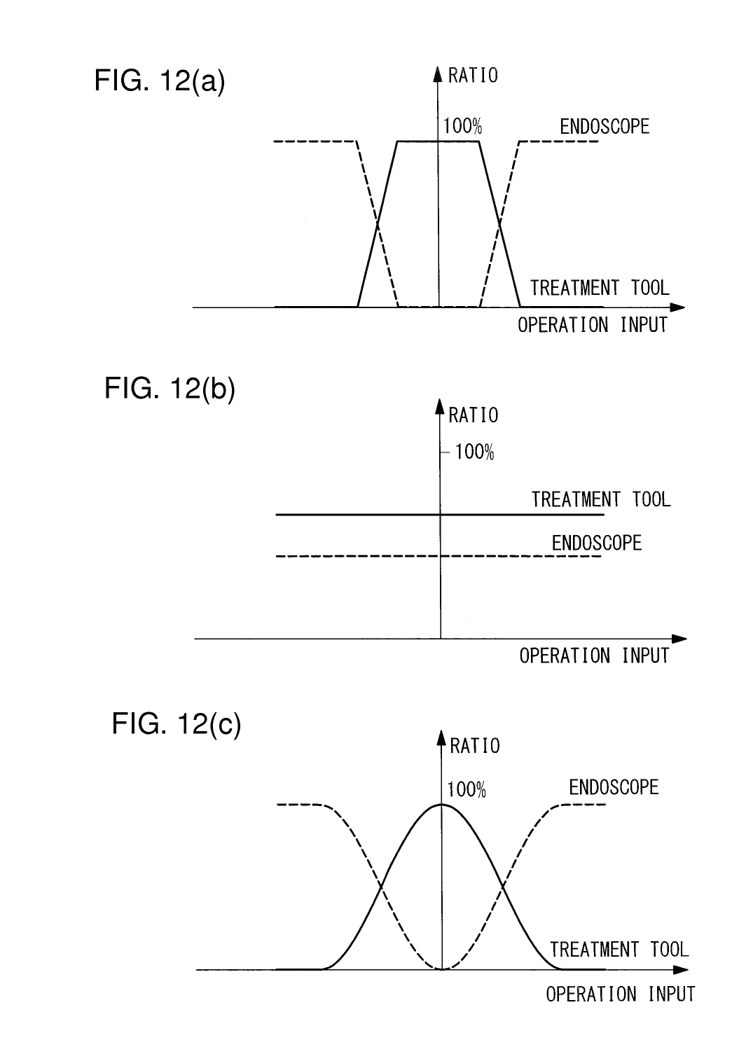

Furthermore, for the above-described embodiment, description is made on the case where switching is performed in accordance with a swiveling angle or the like of the lever 4a of the joystick so as to drive one of the two bending movable portions 2A and 3A. However, without limitation to this, as shown in FIGS. 12(a), 12(b) and 12(c), moving ratios of the two bending movable portions 2A and 3A may be changed.

FIG. 12(a) shows an example of a case where an operating ratio of the second bending movable portion 3A of the treatment tool 3 is 100% in a range in which an operational amount, i.e., a swiveling angle of the lever 4a of the joystick is small, and an operating ratio is gradually and linearly switched from the second bending movable portion 3A of the treatment tool 3 to the first bending movable portion 2A of the endoscope 2 in accordance with increase in a swiveling angle of the lever 4a so that an operating ratio of the first bending movable portion 2A of the endoscope 2 becomes 100% when a swiveling angle of the lever 4a becomes large.

FIG. 12(b) shows an example of a case where each of the first and second bending movable portions 2A and 3A is operated at a constant operating ratio regardless of an operational amount, i.e., a swiveling angle of the lever 4a of the joystick.

FIG. 12(c) shows an example of a case where an operating ratio is gradually and non-linearly switched from the second bending movable portion 3A of the treatment tool 3 to the first bending movable portion 2A of the endoscope 2.

The present embodiment may be applied to a case where two or more treatment tools 3 protrude from the distal end surface 2a of the endoscope 2. In this case, to the input IF 4 for operating one of the treatment tools 3, operation of the same one of the treatment tools 3 and the endoscope 2 may be allocated.

Further, in the present embodiment, the treatment tool 3 protrudes from the distal end surface 2a of the endoscope 2. However, instead of this, the treatment tool 3 may protrude from a side surface of the endoscope 2.

For the present embodiment, the bending movable portions 2A and 3A that perform bending motion are described as an example of the movable portion. However, instead of this, a linearly movable portion that performs linear motion may be adopted.

According to the above embodiment, following aspects can be introduced.

An aspect of the present invention provides a medical device comprising: a long main body including a distal end portion; two or more movable portions arranged at the main body or in a vicinity of the distal end portion; a single input interface part operated by an operator; and a control part switching and controlling the respective movable portions in accordance with an operation amount input to the input interface part.

According to this aspect, when an operator operates the single input interface part, the two or more movable portions are respectively or simultaneously driven so that the distal end portion arranged at the main body can be moved relative to an affected part. In this case, an operator can switch and control the two or more movable portions via the control part only by operating the single input interface part. In other words, it becomes unnecessary to operate the two or more movable portions by two or more operating parts, so that troublesomeness of the operation can be resolved

In the above-described aspect, the two or more movable portions may be bending movable portions that perform bending operation, and may be arranged so as to be spaced from each other in a longitudinal direction of the main body.

With this configuration, the distal end portion is made to perform fine motion by the bending movable portion on the more distal end side, and the distal end portion is made to perform relatively large motion by the bending movable portion on the more proximal end side. In other words, only operating the single input interface part enables rough motion and fine motion of the distal end portion provided at the distal end to be performed.

In the above-described aspect, when an operation amount input to the input interface part is smaller than a predetermined threshold, the control part may drive the bending movable portion arranged on a more distal end side, and when an operation amount is equal to or larger than the threshold, the control part may drive the bending movable portion on a more proximal end side.

Thereby, the bending movable portions to be driven are switched from each other in accordance with magnitude of an operation amount input to the single input interface part, and only by operating the single input interface part, rough motion and fine motion of the distal end portion provided at the distal end can be intuitively switched from each other, and can be simply performed.

In the above-described aspect, the distal end portion may be a treatment tool including an end effector at a distal end thereof.

In the above-described aspect, the main body may be an endoscope.

In the above-described aspect, the movable portion of the treatment tool may be driven when an operation amount input to the input interface part is smaller than a predetermined threshold. The movable portion of the endoscope may be driven when an operation amount is equal to or larger than the threshold, and an operation amount is increased. A drive of the movable portion of the endoscope may be made invalid when an operation amount is equal to or larger than the threshold, and an operation amount is decreased.

Thereby, when an operation amount is small, the movable portion of the treatment tool arranged on the distal end side can be driven to move the distal end of the distal end portion by a small amount, and when an operation amount becomes large, the driven portion of the endoscope arranged on the proximal end side can be driven to move the distal end of the distal end portion by a large amount. Thereby, when an operation amount is small, a visual field by the endoscope is fixed, and in this state, the treatment tool can be driven within the visual field. When the treatment tool moves to an end of the visual field, an operation amount is large, and the visual field by the endoscope can be moved in a direction in which the treatment tool has been moved. When an operation amount is returned from a large state to a small state, drive of the movable portion of the endoscope is made invalid so that the treatment tool can be driven within the visual field in a state where the moved visual field is fixed at the moved position.

In the above-described aspect, the control part may switch and control the respective movable portions on the basis of magnitude of a differential value of an operation amount input to the input interface part.

Thereby, when a differential value of an operation amount, i.e., an operation speed is made small, the movable portion on the distal end side is driven so that fine motion can be accomplished, and when an operation speed is made large, the movable portion on the proximal end side is driven so that large motion can be accomplished.

In the above-described aspect, a movable region detecting part that detects a motion limit of the bending movable portion on a more distal end side may be provided, and the control part may control each of the bending movable portions so as to drive the bending movable portion on a distal end side when the motion limit is not detected by the movable region detecting part, and so as to drive the bending movable portion on a more proximal end side when the motion limit is detected by the movable region detecting part.

Thereby, the bending movable portion on the distal end side is driven until the motion limit of the bending movable portion on the distal end side is detected by the movable region detecting part. After the bending movable portion on the distal end side moves beyond the motion limit, the bending movable portion on the proximal end side can be driven. By the single input interface part, the two or more bending movable portion can be switched and operated.

According to another aspect of the present invention, there is provided a control method for a medical device. The control method includes inputting an operation amount to a single input interface part, and switching two or more movable portions in accordance with the operation amount to be driven. The two or more movable portions are provided in a vicinity of a distal end portion of a long main body including the distal end portion.

The present invention provides an advantage in that two or more movable portions can be simply operated by a single operating part, so that troublesomeness of the operation can be resolved.

### REFERENCE SIGNS LIST

A affected part
1 medical device
2 endoscope (main body)
2A first bending movable portion
3 treatment tool (distal end portion)
3a end effector
3A second bending movable portion
4 input IF (input interface part)
5 control part
8 sensor (movable region detecting part)

1. A medical device comprising:
a long main body including a distal end portion;
two or more movable portions arranged at the main body or in a vicinity of the distal end portion;
a single input interface part operated by an operator; and
a control part switching and controlling the respective movable portions in accordance with an operation amount input to the input interface part.

2. The medical device according to claim 1, wherein the two or more movable portions are bending movable portions that perform bending operation, and are arranged so as to be spaced from each other in a longitudinal direction of the main body.

3. The medical device according to claim 2, wherein when an operation amount input to the input interface part is smaller than a predetermined threshold, the control part drives the bending movable portion arranged on a more distal end side, and when an operation amount is equal to or larger than the threshold, the control part drives the bending movable portion on a more proximal end side.

4. The medical device according to claim 1, wherein the distal end portion is a treatment tool including an end effector at a distal end thereof.

5. The medical device according to claim 4, wherein the main body is an endoscope.

6. The medical device according to claim 5, wherein the movable portion of the treatment tool is driven when an operation amount input to the input interface part is smaller than a predetermined threshold,
wherein the movable portion of the endoscope is driven when an operation amount is equal to or larger than the threshold, and an operation amount is increased, and
wherein a drive of the movable portion of the endoscope is made invalid when an operation amount is equal to or larger than the threshold, and an operation amount is decreased.

7. The medical device according to claim 2, wherein the control part switches and controls the respective movable portions on the basis of magnitude of a differential value of an operation amount input to the input interface part.

8. The medical device according to claim 2, comprising a movable region detecting part that detects a motion limit of the bending movable portion on a more distal end side,
wherein the control part controls each of the bending movable portions so as to drive the bending movable portion on a distal end side when the motion limit is not detected by the movable region detecting part, and so as to drive the bending movable portion on a more proximal end side when the motion limit is detected by the movable region detecting part.

9. A control method for a medical device comprising:
inputting an operation amount to a single input interface part, and
switching two or more movable portions in accordance with the operation amount to be driven,
wherein two or more movable portions are provided in a vicinity of a distal end portion of a long main body including the distal end portion.
